(19) United States
(12) Patent Application Publication
(10) Pub. No.: US 2022/0005568 A1
(43) Pub. Date: Jan. 6, 2022

(54) AUTOMATED IDENTIFICATION OF POTENTIAL DRUG SAFETY EVENTS

(71) Applicant: AGIOS PHARMACEUTICALS, INC., Cambridge, MA (US)

(72) Inventors: Wassim Aldairy, Lexington, MA (US); Peter Frederick Hawkins, Cambridge, MA (US); Bryan Stuart Murray, Arlington, MA (US)

(21) Appl. No.: 17/477,745

(22) Filed: Sep. 17, 2021

Related U.S. Application Data
(63) Continuation of application No. 16/360,061, filed on Mar. 21, 2019.
(60) Provisional application No. 62/397,407, filed on Sep. 21, 2016.

(51) Int. Cl. G16H 15/00; G16H 70/40; G16H 10/20; G06F 16/00; G06F 40/247; G06F 40/279

(52) U.S. Cl. CPC G16H 15/00; G16H 70/40; G16H 10/20; G06F 40/284; G06F 40/247; G06F 40/279; G06F 16/00

(57) ABSTRACT

Various embodiments include methods, computer program products and systems for analyzing reported adverse event (AE) data about a pharmaceutical, vaccine or medical device. In some cases, that reported AE data is unstructured. In these cases, a method can include: applying a natural language processing (NLP) filter to the unstructured reported AE data to generate an initial set of reporting codes for the unstructured reported AE data; providing the initial set of reporting codes for review by a healthcare professional, to either verify each of the reporting codes or modify at least one of the reporting codes, and generating a refined set of reporting codes based upon the review; and creating a safety case report linking the pharmaceutical, vaccine or medical device with the refined set of reporting codes. In additional embodiments, the safety report is provided to relevant authorities according to prescribed reporting criteria.

### CROSS-REFERENCE TO RELATED APPLICATION

This application is a continuation filing of co-pending U.S. patent application Ser. No. 16/360,061 (filed Mar. 21, 2019), which claims priority to Patent Cooperation Treaty (PCT) International Application No. PCT/US2017/051259 (filed Sep. 13, 2017), which claims priority to U.S. Provisional Patent Application No. 62/397,407 (filed Sep. 21, 2016), each of which is hereby incorporated by reference in its entirety.

### TECHNICAL FIELD

Aspects of the disclosure relate generally to pharmaceutical (drug), vaccine or medical device data collection, analysis and reporting. More particularly, various aspects of the disclosure relate to analyzing (e.g., drug) testing data to enhance detection of drug safety events, vaccine safety events or medical device safety events (also known as adverse events).

### BACKGROUND

A drug safety event, vaccine safety event or medical device safety event, also termed an adverse event (AE) herein, is any unexpected or undesirable medical occurrence in a patient or clinical investigation subject that has been administered a pharmaceutical product, vaccine or medical device, where the event does not necessarily have a causal relationship with this treatment. An AE can include, for example, unfavorable and unintended signs (including abnormal laboratory findings), symptoms, or diseases temporally associated with the use of a medicinal (or, investigational) product, whether or not related to the medicinal (or, investigational) product.

AEs in patients participating in clinical trials are reported to the study sponsor, and if required by particular jurisdictions, could be reported to a local ethics panel or other authority. Depending upon jurisdictions, adverse events categorized as “serious” (i.e., events resulting in death, illness requiring hospitalization, events deemed life-threatening, events resulting in persistent or significant disability/incapacity, congenital anomaly/birth defect or other medically important condition) must be reported the regulatory authorities immediately. These serious adverse events are referred to as SAEs in many cases. Non-serious AEs, in contrast, can be documented in a periodic (e.g., monthly, annual, etc.) summary and sent to the appropriate regulatory authority. In many circumstances, the trial sponsor collects AE reports from researchers and trial administrators, and notifies all participating administrators (along with pertinent authorities) of those AEs. This process allows for periodic, contemporaneous feedback on issues in the clinical investigation.

AE data can be reported in a number of ways. For instance, some AE data is reported using fillable forms, such as fillable portable document format (PDF) forms, spreadsheets, textual forms or electronic data capture systems (e.g., web-based forms). AE data can also be reported by an administrator or patient via web-based or closed-network portals. Additionally, AE data can be reported via social media, such as in posts, updates or other messages. Further, AE data can be reported orally, in person or via call centers. This voice data, such as call center data, can be logged and stored for later analysis. The forms (e.g., fillable forms, web-based forms, etc.) and call center logs are sent to the study sponsor, who then analyzes the forms and/or logs to extract data about particular AEs, including commonality of signs, symptoms, diseases, etc. and usage of terminology to describe the AEs and related of signs, symptoms, diseases, etc. This process is conventionally performed manually by human users, for example, by reviewing or printing the forms and/or logs and analyzing the text for particular identifiers. The human users then classify the reported AE data according to identification codes for a particular reporting system, and an AE report is provided to the pertinent authority.

For example, in the United States, the Vaccine Adverse Event Reporting System (VAERS) is used to report AE data for immunization therapies. VAERS includes identification codes tied to symptoms, such as fatigue (ID code XXXX), myalgia (ID code XXXY), dysphagia (ID code XXXZ), etc. These identification codes are built from a dictionary, which in this example, can include the Medical Dictionary for Regulatory Activities (MedDRA). The conventional approach requires the user to convert the AE data, which can include unstructured data (e.g., voice-to-text conversion data or free-form text entry) or structured data (e.g., text structured from fillable forms using optical character recognition (OCR)) into code form using the dictionary and objective and subjective rules.

This conventional approach can miss or otherwise discount significant information about patient (subject) signs, symptoms and diseases due to the nature of the manually-applied rules. For example, reported AE data could include a textual narrative describing a set of symptoms (e.g., “hot pain at injection site; fever; fatigue, headache; muscle pain in arm and shoulder . . . ”). The user, in reviewing that narrative, could miss or fail to account for modifying terms (e.g., hot pain) or combination terms (e.g., muscle pain in arm and shoulder). In other cases, reported AE data can be structured such that it creates false positives (e.g., “no numbness, no weakness”), where rules attach to particular terms without noticing contextual modifiers (e.g., “no”). Further, rules, and the users applying such rules, can fail to account for narrative-type data that does not neatly coincide with pre-existing dictionary definitions or codes. In this instance, less technical terms such as “blacking out,” “falling down,” etc. may be incorrectly coded or otherwise ignored in processing reported AE data. Additionally, because AE data for particular patients is logged in distinct time-related entries, the conventional approach does not allow for tracking individual patient progression over a period. That is, a patient may report “minor pain in arm” on day 1, and “severe pain in arm” on day 2, and the conventional approach may merely note the separate occurrences of “pain” without noting the progression from “minor” to “severe” over that period. As such, the conventional approach for processing reported AE data has many shortcomings. This conventional approach can be time consuming, costly, and error-prone.

### BRIEF SUMMARY

Various embodiments of the disclosure include methods, computer program products and systems for analyzing reported adverse event (AE) data about a pharmaceutical or other medial implementation subject to regulatory approval and/or reporting (e.g., a vaccine or medical device such as an implantable device, wearable medical device or external medical device). In some cases, that reported AE data is unstructured. In these cases, a method can include: applying a natural language processing (NLP) filter to the unstructured reported AE data to generate an initial set of reporting codes for the unstructured reported AE data; providing the initial set of reporting codes for review by a healthcare professional, to either verify each of the reporting codes or modify at least one of the reporting codes, and generating a refined set of reporting codes based upon the review; and creating a safety case report linking the pharmaceutical or other medical implementation with the refined set of reporting codes. In additional embodiments, the safety report is provided to relevant authorities according to prescribed reporting criteria.

Some particular aspects of the disclosure include a computer program product having program code, which when executed on at least one computing device, causes the at least one computing device to analyze unstructured reported adverse event (AE) data about a pharmaceutical or other medical implementation by performing actions including: applying a natural language processing (NLP) filter to the unstructured reported AE data to generate an initial set of reporting codes for the unstructured reported AE data; providing the initial set of reporting codes for review by a healthcare professional, to either verify each of the reporting codes or modify at least one of the reporting codes, and generating a refined set of reporting codes based upon the review; and creating a safety case report linking the pharmaceutical or other medical implementation with the refined set of reporting codes.

Various additional aspects of the disclosure include a system having: at least one computing device configured to analyze unstructured reported adverse event (AE) data about a pharmaceutical or other medical implementation by performing actions including: applying a natural language processing (NLP) filter to the unstructured reported AE data to generate an initial set of reporting codes for the unstructured reported AE data; providing the initial set of reporting codes for review by a healthcare professional, to either verify each of the reporting codes or modify at least one of the reporting codes, and generating a refined set of reporting codes based upon the review; and creating a safety case report linking the pharmaceutical or other medical implementation with the refined set of reporting codes.

Other aspects of the disclosure include a computer-implemented method for analyzing structured reported adverse event (AE) data about a pharmaceutical or other medical implementation, the method including: applying optical character recognition (OCR) to the structured reported AE data to generate an initial set of reporting codes for the structured reported AE data; providing the initial set of reporting codes for review by a healthcare professional, to either verify each of the reporting codes or modify at least one of the reporting codes, and generating a refined set of reporting codes based upon the review; and creating a safety case report linking the pharmaceutical or other medical implementation with the refined set of reporting codes.

Further aspects of the disclosure include a computer program product having program code, which when executed on at least one computing device, causes the at least one computing device to analyze structured reported adverse event (AE) data about a pharmaceutical or other medical implementation by performing actions including: applying optical character recognition (OCR) to the structured reported AE data to generate an initial set of reporting codes for the structured reported AE data; providing the initial set of reporting codes for review by a healthcare professional, to either verify each of the reporting codes or modify at least one of the reporting codes, and generating a refined set of reporting codes based upon the review; and creating a safety case report linking the pharmaceutical or other medical implementation with the refined set of reporting codes.

Additional aspects of the disclosure include a system having: at least one computing device configured to analyze structured reported adverse event (AE) data about a pharmaceutical or other medical implementation by performing actions including: applying optical character recognition (OCR) to the structured reported AE data to generate an initial set of reporting codes for the structured reported AE data; providing the initial set of reporting codes for review by a healthcare professional, to either verify each of the reporting codes or modify at least one of the reporting codes, and generating a refined set of reporting codes based upon the review; and creating a safety case report linking the pharmaceutical or other medical implementation with the refined set of reporting codes.

Other aspects of the disclosure include a computer-implemented method for analyzing unstructured reported adverse event (AE) data about a pharmaceutical or other medical implementation, the method including: applying a natural language processing (NLP) filter to the unstructured reported AE data to generate an initial set of reporting codes for the unstructured reported AE data; applying a data visualization filter to the set of reporting codes to create a visual depiction of the reporting codes for the unstructured reported AE data; providing the visual depiction for review by a healthcare professional, to either verify each of the reporting codes or modify at least one of the reporting codes, and generating a refined set of reporting codes based upon the review; and creating a safety case report linking the pharmaceutical or other medical implementation with the refined set of reporting codes.

Further aspects of the disclosure include a computer program product having program code, which when executed on at least one computing device, causes the at least one computing device to analyze unstructured reported adverse event (AE) data about a pharmaceutical or other medical implementation by performing actions including: applying a natural language processing (NLP) filter to the unstructured reported AE data to generate an initial set of reporting codes for the unstructured reported AE data; applying a data visualization filter to the set of reporting codes to create a visual depiction of the reporting codes for the unstructured reported AE data; providing the visual depiction for review by a healthcare professional, to either verify each of the reporting codes or modify at least one of the reporting codes, and generating a refined set of reporting codes based upon the review; and creating a safety case report linking the pharmaceutical or other medical implementation with the refined set of reporting codes.

Additional aspects of the disclosure include a system having: at least one computing device configured to analyze unstructured reported adverse event (AE) data about a pharmaceutical or other medical implementation by performing actions including: applying a natural language processing (NLP) filter to the unstructured reported AE data to generate an initial set of reporting codes for the unstructured reported AE data; applying a data visualization filter to the set of reporting codes to create visual depiction of the reporting codes for the unstructured reported AE data; providing the visual depiction for review by a healthcare professional, to either verify each of the reporting codes or modify at least one of the reporting codes, and generating a refined set of reporting codes based upon the review; and creating a safety case report linking the pharmaceutical or other medical implementation with the refined set of reporting codes.

### BRIEF DESCRIPTION OF THE DRAWINGS

FIG. 1 shows a schematic depiction of a computing environment for providing an adverse event data analysis system according to various embodiments of the disclosure.

FIG. 2 shows a schematic depiction of a data-process flow according to various embodiments of the disclosure.

FIG. 3 is a flow diagram detailing processes performed in the data-process flow diagram of FIG. 2.

FIG. 4 shows an example table illustrating reported unstructured adverse event data.

FIG. 5 shows an example table illustrating adverse event data for a subject at distinct time intervals.

FIG. 6 shows a schematic depiction of a data-process flow according to various additional embodiments of the disclosure.

FIG. 7 is a flow diagram detailing processes performed in the data-process flow diagram of FIG. 6.

FIG. 8 shows an example depiction of structured reported adverse event data, in the form of a section from a fillable severe adverse event (SAE) reporting form used according to various embodiments of the disclosure.

FIG. 9 shows a schematic depiction of a data-process flow according to various other embodiments of the disclosure.

FIG. 10 is a flow diagram detailing processes performed in the data-process flow diagram of FIG. 9.

FIG. 11 shows an example visual depiction of reporting codes for adverse event data, generated according to embodiments of the disclosure.

FIG. 12 shows an example visual depiction of reporting codes for adverse event data, generated according to embodiments of the disclosure.

It is noted that the drawings of the disclosure are not necessarily to scale. The drawings are intended to depict only typical aspects of the disclosure, and therefore should not be considered as limiting the scope of the disclosure. In the drawings, like numbering represents like elements between the drawings.

### DETAILED DESCRIPTION

This disclosure relates generally to pharmaceutical (drug), vaccine and/or medical device trial reporting. More particularly, various aspects of the disclosure relate to systems, computer program products, and methods for analyzing drug, vaccine and/or medical device trial data to detect drug, vaccine and/or medical device safety events (also known as adverse events, or AEs).

According to various embodiments, the processes, systems and computer program products described herein may be used in other systems, e.g., network analysis tools, or in other forms of data analysis and reporting. For example, the approaches described herein could be applied to any other medial implementation subject to regulatory approval and/or reporting (e.g., a vaccine or medical device such as an implantable device, wearable medical device or external medical device).

As noted herein, conventional approaches for processing reported AE data are prone to error, time-consuming and costly. Embodiments of the present disclosure are directed to automated systems and related approaches for analyzing reported adverse event data. In particular, these approaches are configured to reduce the time and expense of processing reported AE data by orders of magnitude.

In one embodiment, a process includes: i) applying a natural language processing (NLP) filter to unstructured (reported) AE data (e.g., a text string, social media data, etc.) for a pharmaceutical, vaccine or medical device to generate an initial set of reporting codes for the unstructured AE data; ii) reviewing, by a healthcare professional, the initial set of reporting codes to either verify each of those reporting codes or modify at least one of the reporting codes and generate a refined set of reporting codes; iii) creating a safety case report linking the pharmaceutical, vaccine or medical device with the refined (or initial, if not modified) set of reporting codes; and iv) providing the safety case report, e.g., to a regulatory or other authority.

In many cases, the above-noted process is repeated for a pool of subjects (e.g., one or more subjects, or patients), and tracks progression for each subject over time. That is, an AE report for Patient 1, having a unique patient identifier, can be generated at distinct times (t₁, t₂, t₃) and automatically compared with other AE reports for that subject. In various embodiments, only the data that has changes for Subject 1 from t₁ to t₂, or t₂ to t₃, etc., is identified, streamlining entries for review by the healthcare professional.

In various embodiments, the NLP filter can include a conventional NLP algorithm and an adverse event thesaurus (AE thesaurus) that can be iteratively refined using results from each pass through the NLP filter. That is, over time, the NLP filter will continue to develop additional thesaurus terms and filter rules for processing reported AE data. Additionally, the AE thesaurus can be manually updated and/or refined as new terms and correlations are made available.

In another embodiment, a process includes: i) applying optical character recognition (OCR) to structured (reported) AE data (e.g., fillable PDF text data) for a pharmaceutical, vaccine or medical device to generate an initial set of reporting codes for the unstructured AE data; ii) reviewing, by a healthcare professional, the initial set of reporting codes to either verify each of those reporting codes or modify at least one of the reporting codes and generate a refined set of reporting codes; iii) creating a safety case report linking the pharmaceutical, vaccine or medical device with the refined (or initial, if not modified) set of reporting codes; and iv) providing the safety case report, e.g., to a regulatory or other authority.

In yet another embodiment, a process includes: i) applying a natural language processing (NLP) filter to unstructured (reported) AE data (e.g., a text string, social media data, etc.) for a pharmaceutical, vaccine or medical device to generate an initial set of reporting codes for the unstructured AE data; ii) apply a data visualization filter to the reporting codes to create a (e.g., three-dimensional (3D)) visual depiction of the reporting codes for each patient; iii) reviewing, by a healthcare professional, the visual depiction to either verify each of the reporting codes or modify at least one of the reporting codes and generate a refined set of reporting codes; iv) creating a safety case report linking the pharmaceutical, vaccine or medical device with the refined (or initial, if not modified) set of reporting codes; and v) providing the safety case report, e.g., to a regulatory or other authority.

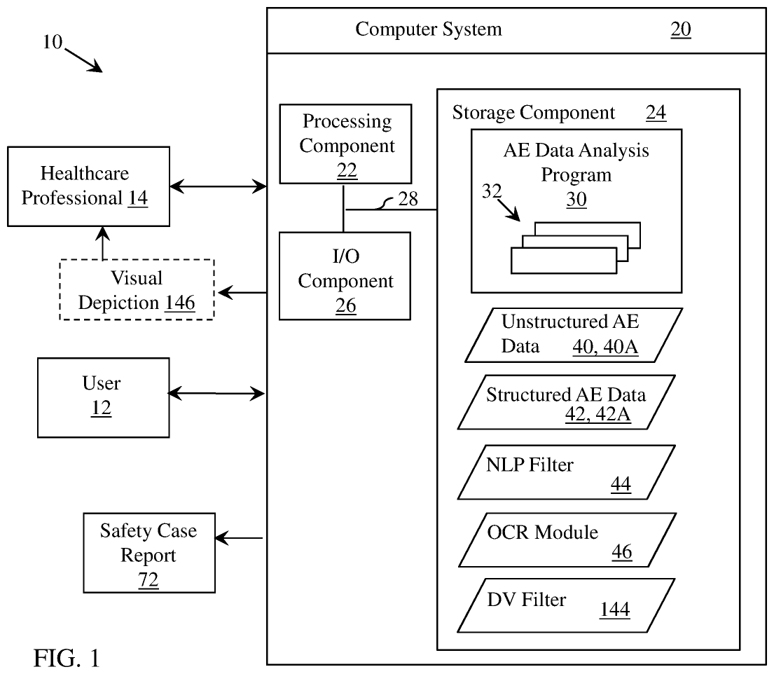

Turning to the drawings, FIG. 1 shows an illustrative environment 10 for performing adverse event (AE) data analysis functions according to an embodiment of the disclosure. To this extent, environment 10 includes a computer system 20 that can perform one or more processes described herein in order to analyze reported AE data. In particular, computer system 20 is shown including an adverse event (AE) data analysis program 30, which makes computer system 20 operable to analyze reported AE data by performing a process described herein.

Computer system 20 is shown including a processing component 22 (e.g., one or more processors), a storage component 24 (e.g., a storage hierarchy), an input/output (I/O) component 26 (e.g., one or more I/O interfaces and/or devices), and a communications pathway 28. In general, processing component 22 executes program code, such as AE data analysis program 30, which is at least partially fixed in storage component 24. While executing program code, processing component 22 can process data, which can result in reading and/or writing transformed data from/to storage component 24 and/or I/O component 26 for further processing. Pathway 28 provides a communications link between each of the components in computer system 20. I/O component 26 can comprise one or more human I/O devices, which enable a human user 12 and/or a healthcare professional 14 to interact with computer system 20 and/or one or more communications devices to enable system user 12 and/or healthcare professional 14 to communicate with computer system 20 using any type of communications link. It is understood that as used herein, the term “healthcare professional” can refer to a human being (human user), or to a programmable computing device including a logic engine, e.g., to make healthcare decisions as described herein. When healthcare professional 14 is a human being (e.g., human user), the term may refer to a qualified healthcare professional such as a doctor/physician, nurse, nurse practitioner, physician assistant, pharmacist, nutritionist, etc. A healthcare professional 14 can also include any other trained professional working in concert with or under supervision of a qualified healthcare professional (such as those noted above). These trained professionals could include a scientist, a data analyst, a data scientist, a safety scientist, a global product specialist, etc.

AE data analysis program 30 can manage a set of interfaces (e.g., graphical user interface(s), application program interface, and/or the like) that enable human and/or system users 12, as well as healthcare professional(s) 14, to interact with AE data analysis program 30. Further, AE data analysis program 30 can manage (e.g., store, retrieve, create, manipulate, organize, present, etc.) data, and files, such as unstructured AE data 40, structured AE data 42, natural language processing (NLP) filter 44, optical character recognition (OCR) module 46 and/or data visualization (DV) filter 144 using any solution.

In various embodiments, unstructured AE data 40 can include data about a sign, symptom or disease of a clinical trial subject (e.g., a patient or other trial participant), or post-marketing data such as social media data or published literature (e.g., articles, journal findings or reviews) about a pharmaceutical, vaccine or medical device. In particular cases, the unstructured reported AE data 40 includes information that does not have a pre-defined data model, or is not organized in a pre-defined manner While this unstructured (reported) AE data 40 may be primarily textual data, it may include data such as dates, numbers, and facts. In some cases, unstructured AE data 40 includes a string of text, a social media post, or a voice-to-text conversion of an audio recording.

In various embodiments, structured (reported) AE data 42 includes information with a high degree of organization, for instance, such that the structured AE data 42 could be readily searchable using simple search engine algorithms or other search operations. This structured AE data 42 could be presented in column/row form or in another format that is easily integrated into a relational database. Like unstructured AE data 40, structured AE data 42 includes data about a sign, symptom or disease of a clinical trial subject. In some particular cases, the structured AE data 42 includes a fillable portable document format (PDF) file, an entry in a spreadsheet, or a fillable text form.

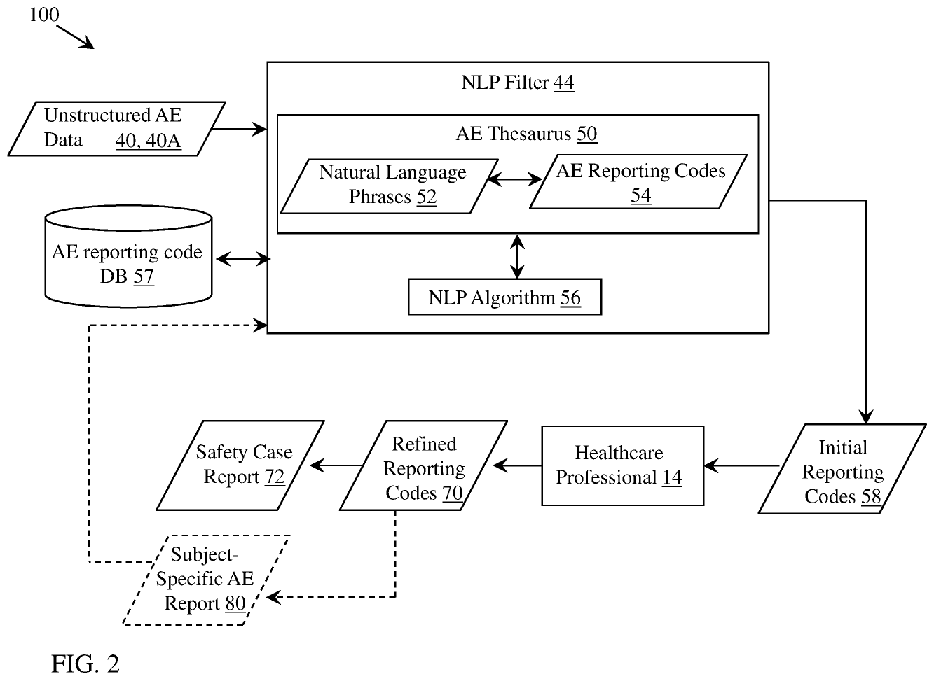

In various embodiments, the NLP filter 44 includes an adverse event thesaurus (AE thesaurus) 50 having correlations between natural language phrases 52 and AE reporting codes 54 (illustrated in data flow in FIG. 2). Further, NLP filter 44 can include an NLP algorithm 56 configured to perform at least one of the following to the unstructured reported AE data 40 to generate an initial set of reporting codes 58: ESG parsing, entity detection, sense disambiguation, aggregation, declarative rule generation, relationship extraction, sentence breaking or word segmentation. In some cases, NLP filter 44 (including NLP algorithm 56) can be configured to perform one or more of the above-noted NLP techniques to unstructured reported AE data 40, e.g., from what is known in the art as “organized data collection systems” or the like. For example, as defined in Section VI.B.1.2. (Solicited Reports) of the European Medicines Agency's Guidelines on good pharmacovigilance practices (GVP), “solicited reports of suspected adverse reactions are those derived from organised data collection systems, which include clinical trials, non-interventional studies, registries, post-approval named patient use programmes, other patient support and disease management programmes, surveys of patients or healthcare providers, compassionate use or name patient use, or information gathering on efficacy or patient compliance. Reports of suspected adverse reactions obtained from any of these data collection systems should not be considered spontaneous.”

As described herein, the AE thesaurus 50 within NLP filter 44 is configured to add new natural language phrases 52 and correlations with AE reporting codes 54 iteratively, i.e., as AE data analysis program 30 processes data such as unstructured AE data 40. In some cases, AE thesaurus 50 is manually updateable, e.g., by a user 12, to implement new correlations between natural language phrase 52 and reporting codes 54.

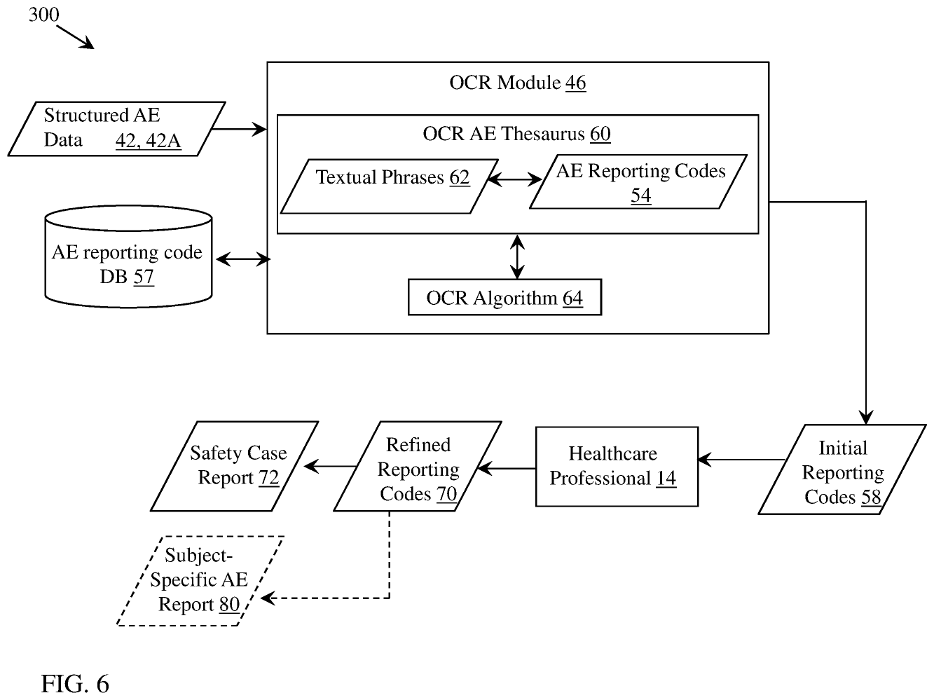

OCR module 46 can also include an adverse event thesaurus (AE thesaurus), which may overlap with or include AE thesaurus 50 used in NLP filter 44, or may include a distinct OCR-specific AE thesaurus 60 (FIG. 6). The OCR-specific AE thesaurus 60 can include correlations between text (and textual phrases) 62 and reporting codes 54. OCR module 46 can include an OCR algorithm 64 configured to perform at least one of the following to the structured reported AE data 42 to generate the initial set of reporting codes 58: desquew, despeckle, script rules, text string search, check mark (including check mark group recognition), row recognition, etc. In various embodiments, OCR module 46 can obtain the structured reported AE data 42, rotate, desquew and/or despeckle the AE data 42, and then apply script rules (e.g., from AE thesaurus 60) based upon the headers, footers and/or images on the intake forms. In various embodiments, OCR module 46 can identify particular terms and data categories using text string search, check mark and check mark group recognition, and/or repeating row recognition (e.g., for tables). Additionally, OCR module 46 can identify a known point or heading in the AE data 42 as an indicator of input terms or characters, e.g., below, above or on a side of the data input. These terms can be matched with the reporting codes 58 according to OCR rules (e.g., in OCR algorithm 64).

Data visualization (DV) filter 144 can include any data visualization software capable of converting unstructured AE data 40 to a visual depiction 146, which may be presented to healthcare professional 14 as described herein. In some cases, visual depiction 146 includes a three-dimensional data map, or cluster map, emphasizing the interconnections between particular AE signs, symptoms and/or diseases and particular subject(s) or their groups. In other cases, visual depiction 146 can include a “heat map” of unstructured AE data 40, indicating intensity of occurrences of particular signs, symptoms and/or disease. In some cases, DV filter 144 can utilize open-source software such as Cytoscape, or a proprietary software system, to generate one or more visual depiction(s) 146 of unstructured AE data 40.

With continuing reference to FIG. 1, in any event, computer system 20 (including AE data analysis program 30) can obtain unstructured AE data 40, structured AE data 42, NLP filter 44 and/or OCR module 46, using any solution. For example, computer system 20 can generate and/or be used to generate unstructured AE data 40, structured AE data 42, NLP filter 44 and/or OCR module 46, retrieve unstructured AE data 40, structured AE data 42, NLP filter 44 and/or OCR module 46 from one or more data stores, receive unstructured AE data 40, structured AE data 42, NLP filter 44 and/or OCR module 46 from another system, and/or the like.

Computer system 20 can comprise one or more general purpose computing articles of manufacture (e.g., computing devices) capable of executing program code, such as AE data analysis program 30, installed thereon. As used herein, it is understood that “program code” means any collection of instructions, in any language, code or notation, that cause a computing device having an information processing capability to perform a particular action either directly or after any combination of the following: (a) conversion to another language, code or notation; (b) reproduction in a different material form; and/or (c) decompression. To this extent, AE data analysis program 30 can be embodied as any combination of system software and/or application software.

Further, AE data analysis program 30 can be implemented using a set of modules 32. In this case, a module 32 can enable computer system 20 to perform a set of tasks used by AE data analysis program 30, and can be separately developed and/or implemented apart from other portions of AE data analysis program 30. As used herein, the term “component” means any configuration of hardware, with or without software, which implements the functionality described in conjunction therewith using any solution, while the term “module” means program code that enables a computer system 20 to implement the actions described in conjunction therewith using any solution. When fixed in a storage component 24 of a computer system 20 that includes a processing component 22, a module is a substantial portion of a component that implements the actions. Regardless, it is understood that two or more components, modules, and/or systems may share some/all of their respective hardware and/or software. Further, it is understood that some of the functionality discussed herein may not be implemented or additional functionality may be included as part of computer system 20.

When computer system 20 comprises multiple computing devices, each computing device can have only a portion of AE data analysis program 30 fixed thereon (e.g., one or more modules 32). However, it is understood that computer system 20 and AE data analysis program 30 are only representative of various possible equivalent computer systems that may perform a process described herein. To this extent, in other embodiments, the functionality provided by computer system 20 and AE data analysis program 30 can be at least partially implemented by one or more computing devices that include any combination of general and/or specific purpose hardware with or without program code. In each embodiment, the hardware and program code, if included, can be created using standard engineering and programming techniques, respectively.

Regardless, when computer system 20 includes multiple computing devices, the computing devices can communicate over any type of communications link. Further, while performing a process described herein, computer system 20 can communicate with one or more other computer systems using any type of communications link. In either case, the communications link can comprise any combination of various types of optical fiber, wired, and/or wireless links; comprise any combination of one or more types of networks; and/or utilize any combination of various types of transmission techniques and protocols.

As discussed herein, the AE data analysis program 30 enables computer system 20 to analyze unstructured AE data 40 and/or structured AE data 42 according to the various embodiments of the disclosure. Various distinct approaches are disclosed according to embodiments of the disclosure, and for clarity of illustration, these approaches are separated by section headings. It is understood that aspects of particular approaches may be performed in other methods, and that many processes described according to one approach may be combined and/or modified to fit other particular approaches.

Analyzing Unstructured AE Data using NLP

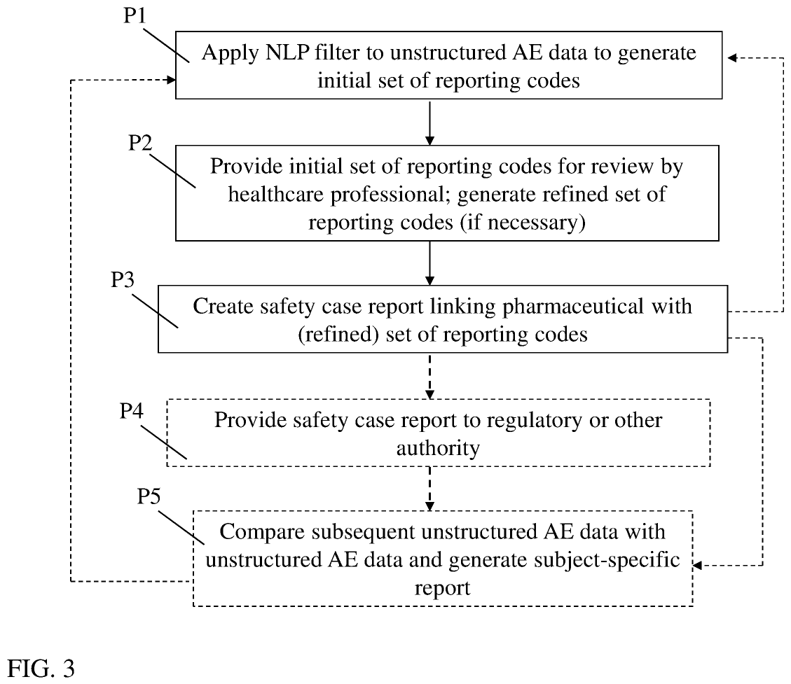

Turning to FIG. 2, a schematic data flow diagram 100 illustrating functions performed by the AE data analysis program 30 is shown according to various embodiments of the disclosure. FIG. 3 is a flow diagram illustrating processes performed in the data flow diagram 100 of FIG. 2. Dashed lines in flow diagrams may indicate optional processes, or those performed according to various distinct embodiments. Processes in the flow diagrams may be combined, re-ordered, and/or modified and still remain within the various aspects of the disclosure. Referring to FIGS. 2 and 3 simultaneously, AE data analysis program 30 is configured to perform processes including:

Process P1: applying natural language processing (NLP) filter 44 to the unstructured reported AE data 40 to generate an initial set of reporting codes 58 for that unstructured reported AE data 40. As noted herein, the NLP filter 44 can include the adverse event thesaurus (AE thesaurus) 50 having correlations between natural language phrases 52 and AE reporting codes 54 (illustrated in data flow in FIG. 2). AE thesaurus 50 can include internally managed connections between natural language phrases 52 and AE reporting codes 54, and can be updated continuously based upon results returned from NLP algorithm 56 running unstructured AE data 40, or manual input from a user (e.g., user 12). Additionally, in various embodiments, AE thesaurus 50 can pull AE reporting codes 54 from an AE reporting code database (DB) 57. AE reporting code DB 57 can include reporting codes from one or more authorities and/or agencies affiliated with reporting of adverse events for pharmaceuticals, vaccines or medical devices. For example, AE reporting code DB 57 can include one or more MedDRA databases, VAERS databases, or other verified databases linking AE reporting codes 54 with particular signs, symptoms or diseases. AE thesaurus 50 can be configured to send updates to AE reporting code DB 57 continuously, periodically or on-demand In various embodiments, a copy of AE reporting code DB 57 can be locally stored at computer system 20, and may be periodically updated. In other cases, AE reporting code DB 57 can be accessed at a central or remote location, where it remains continuously, or periodically, updated.

Further, as noted herein, NLP filter 44 can include an NLP algorithm 56 configured to perform at least one of the following to the unstructured reported AE data 40 to generate an initial set of reporting codes 58: English slot grammar (ESG) parsing, entity detection, sense disambiguation, aggregation, declarative rule generation, relationship extraction, sentence breaking or word segmentation. In some cases, as noted herein, NLP filter 44 (including NLP algorithm 56) can be configured to perform one or more of the above-noted NLP techniques to unstructured reported AE data 40, e.g., from what is known in the art as “organized data collection systems” or the like, such as defined in Section VI.B.1.2. (Solicited Reports) of the European Medicines Agency's Guidelines on good pharmacovigilance practices (GVP), as discussed above.

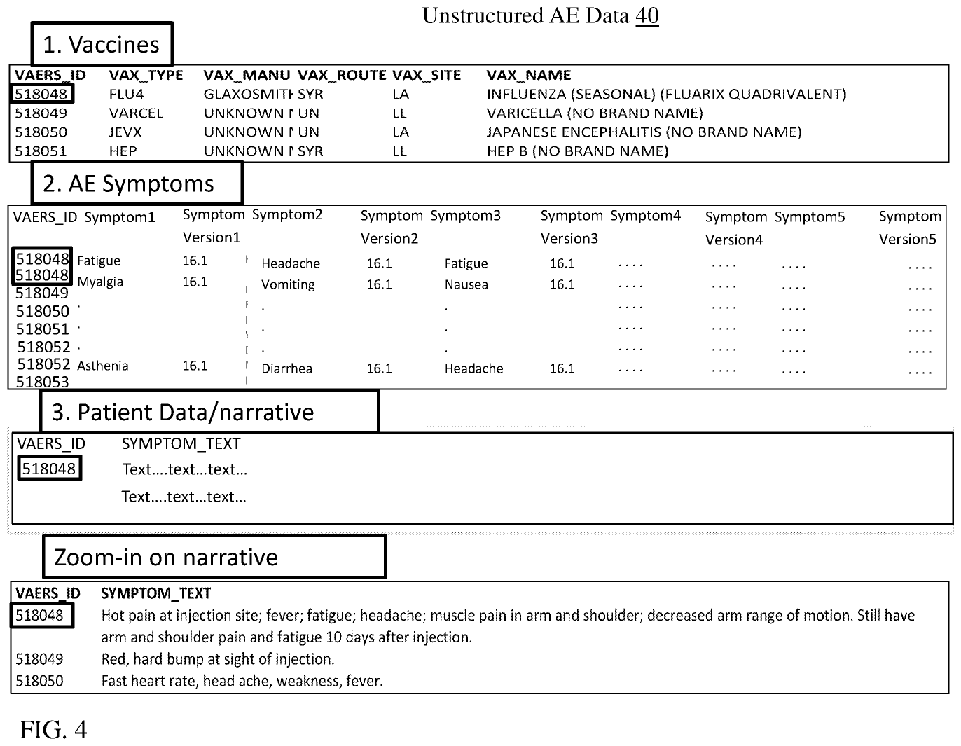

As noted herein, unstructured AE data 40 can include data about a sign, symptom or disease of a clinical trial subject (e.g., a patient or other trial participant), or post-marketing data such as social media data or published literature (e.g., articles, journal findings or reviews) about a pharmaceutical, vaccine or medical device. In particular cases, the unstructured reported AE data 40 includes information that does not have a pre-defined data model, or is not organized in a pre-defined manner While this unstructured (reported) AE data 40 may be primarily textual data, it may include data such as dates, numbers, and facts. That is, in some cases, unstructured AE data 40 includes a string of text, a social media post, or a voice-to-text conversion of an audio recording. FIG. 4 shows an example depiction of unstructured reported AE data 40, in the form of VAERS (vaccine event adverse reporting) data for particular vaccines. As shown, the VAERS data is divided into three data files: 1. Vaccines; 2. Adverse Event Symptoms; and 3. Patient data/narrative. In particular, it is clear that the patient narrative portion of this unstructured reported AE data 40 includes natural language phrases which may not neatly coincide with predefined reporting codes. For example, as noted herein, terms in the narrative, “hot pain at injection site; fever; fatigue; muscle pain in arm and shoulder; decreased arm range of motion; Still have arm and shoulder pain and fatigue 10 days after injection,” can be misreported or otherwise overlooked in conventional approaches. For example, the underlined term “hot” may be parsed from “pain” and fail to accurately describe the type of pain that the patient endures. NLP filter 44 is configured to identify the natural language context of “hot pain” and call for a separate AE reporting code 54 and/or flag this AE reporting code 54 for follow-up by healthcare professional 14 in the set of initial reporting codes 58. Further, the term “and,” separating “arm” from “shoulder,” indicates that the muscle pain is present in both body parts. NLP filter 44 is configured to identify the natural language context of this phrase and select AE reporting codes 54 for both muscle pain in the arm and muscle pain in the shoulder. Additionally, NLP filter 44 can identify the natural language context of the phrase “still have arm and shoulder pain and fatigue 10 days after injection,” and select AE reporting codes 54 indicating prolonged pain in the arm after injection, prolonged pain in the shoulder after injection, prolonged fatigue in the arm after injection and prolonged fatigue in the shoulder after injection. As noted further herein, NLP filter 44 can also flag time-related AE reporting codes 54 for review with subsequent (or prior) unstructured AE data 40 in order to compare the progress of particular signs, symptoms and diseases for a subject.

While VAERS data is used as an example illustration of unstructured reported AE data 40, it is understood that this data may take many forms. Unstructured reported AE data 40 can include a string of text (e.g., provided in a patient log or online portal), a phrase in an online forum, a voice-to-text conversion, a social media post, or post-marketing data such published literature (e.g., articles, journal findings or reviews) about a pharmaceutical, vaccine or medical device. For example, unstructured reported AE data 40 could include a string of text from a patient log which reads, “shoulder pain, scapular region, no numbness weakness.” As noted herein, conventional methods for reviewing this data are prone to error and labor-intensive. The NLP filter 44, however, is configured to process this string of natural language text and determine that the shoulder pain occurs in the scapular region, despite the use of the comma to separate “pain” and “scapular.” Further, NLP filter 44 is configured to determine that there is no numbness and no weakness based upon the syntax of the description (e.g., no separating punctuation between “numbness” and “weakness”, and conventional use of negation phrases at the end of descriptions). In other cases, the unstructured reported AE data 40 could take the form of a social media feed, such as a post or SMS-style message, e.g., “took med. X today and have been dragging ever since.” NLP filter 44 can identify the medication (med X.), time frame (comparing timestamp with term “today”), and the symptom (fatigue, as a close corollary with “dragging”) from this social media data and assign one or more AE reporting codes 54.

NLP filter 44 is also configured to assign a confidence score in its matching of natural language phrases 52 with AE reporting codes 54. That is, according to various embodiments, NLP algorithm 56 may have scores assigned to particular relationships between natural language terms and symptoms. For example, a term such as “dragging,” could be tied with “fatigue,” but could also be tied with “drowsiness.” As such, a code match for “dragging” with the symptom Fatigue could be given a lower confidence score than a code match for “exhausted” with Fatigue. A term such as “sleepy” could have a higher confidence score for the symptom Drowsiness than would the term “dragging.” These confidence scores can be indicated in the initial reporting codes 58, and certain threshold confidence scores (e.g., below level X) can be flagged for additional or special review by healthcare professional 14. In various embodiments, NLP algorithm 56 can take the form of a machine learning algorithm, e.g., a decision tree, naïve Bayesian algorithm and/or a logit algorithm.

Returning to FIGS. 2 and 3, following process P1, process P2 can include: providing the initial set of reporting codes 58 for review by a healthcare professional 14, to either verify each of the reporting codes 58 or modify at least one of the reporting codes 58, and generating a refined set of reporting codes 70 based upon the review. In various embodiments, providing the initial set of reporting codes 58 includes displaying, sending or presenting an editable version of the initial set of reporting codes 58 to the healthcare professional 14. As noted with respect to process P1, particular reporting codes 54 in the set of initial reporting codes 58 can be flagged for follow-up attention by the healthcare professional 14. These codes 54 may include those codes generated by NLP filter 44 in analyzing natural language phrases, such as those illustrated with respect to FIG. 4. The healthcare professional 14 can review this initial set of codes 58, via a user interface, software program, or in another interactive format, and update and/or edit the initial set of codes 58 based upon that professional's judgment. These modifications can be made, for example, via the user interface, software program, or by hand. Generating the refined set of reporting codes 70 can include incorporating at least one modification from the initial set of codes 58 based upon an edit made by the healthcare professional 14. As noted herein, the healthcare professional 14 may take the form of a human user, in which case this process of providing the initial set of reporting codes 58 can include providing a user interface (e.g., via I/O component 26) to output (e.g., display or otherwise present) the initial set of reporting codes 58 for the healthcare professional 14 to review. This user interface could include any conventional interface for providing interaction with a human user, e.g., a touch screen, control system/device (e.g., controller), a wearable system or device, etc. In the case that the healthcare professional 14 includes a computing device (e.g., a computer system having a logic engine), the process of providing the initial set of reporting codes 58 can include transmitting or otherwise making available a data file including the initial set of reporting codes 58 for analysis by the healthcare professional 14. In these cases, healthcare professional 14 can be programmed or otherwise configured to analyze the initial set of reporting codes 58 using a healthcare professional algorithm (and in some cases, a database and/or decision engine) including logic for making decisions regarding the appropriateness of the codes and other information within the initial set of reporting codes 58 as it relates to particular patients, pharmaceuticals, vaccines, medical device etc.

After generating the refined set of reporting codes 70, process P3 can include: creating a safety case report 72 linking the pharmaceutical, vaccine or medical device with the refined set of reporting codes 70. The safety case report 72 can include individual subject reporting codes, as well as codes sorted according to severity, frequency, geography or any other pertinent sorting/grouping criteria. Additionally, safety case report 72 can include a narrative of the course of the (adverse) event, a medical history of the subject, concomitant medications with the pharmaceutical, an assessment (e.g., from event reporter) of causality, and/or an assessment (e.g., from event reporter or other source) as to whether the event is expected as per the product label.

In various embodiments, the process can further include:

Process P4: providing the safety case report 72 to a regulatory authority or other authority. In some cases, the safety case report 72 is provided to a third party or other central body, which may subsequently provide that report 72 to a regulatory or other authority. In other cases, the safety case report 72 is provided directly to the regulatory authority or other authority according to a prescribed schedule, e.g., immediately for severe AEs, and periodically for non-severe AEs. Safety case report 72 can be uploaded or otherwise entered through a secure portal or network connected with the regulatory or other authority.

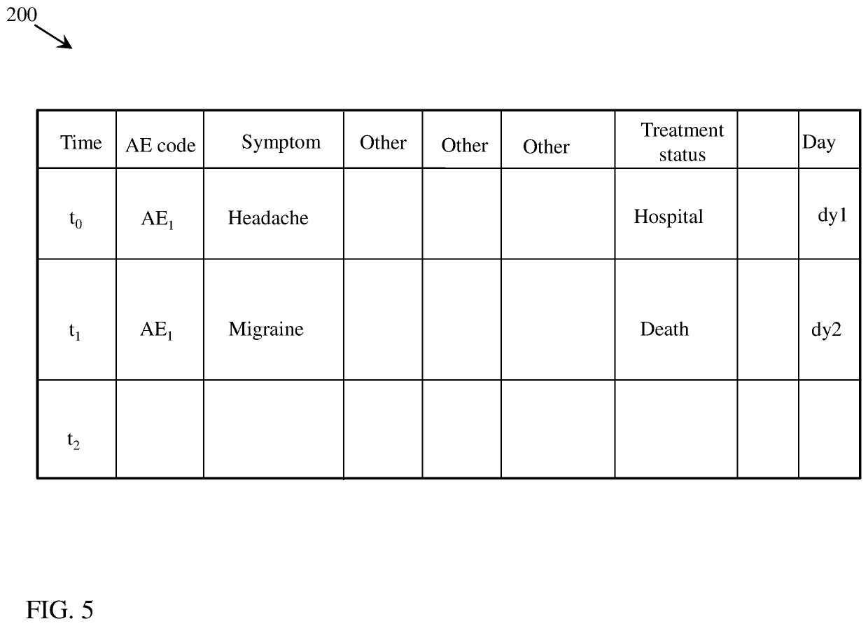

Additionally, as shown in FIG. 3, in some cases, processes P1-P3 can be repeated for subsequent unstructured reported AE data 40A. This subsequent unstructured reported AE data 40A, along with the unstructured AE data 40 each include subject-specific AE data about a set of trial subjects. In some cases, the subsequent unstructured reported AE data 40A describes a sign, symptom or disease of the set of subjects in response to the pharmaceutical, vaccine or medical device at a time (t₁) later than the unstructured reported AE data 40 (from time t₀) about the subject. FIG. 5 shows an example table 200 depicting a portion of subject-specific AE data (i.e., data about a particular trial subject) from unstructured reported AE data 40 (at time t₀) and subsequent unstructured reported AE data 40A (at time t₁). This data indicates that a subject at time t₀reported a headache, coded as an AE1, and was admitted to, or treated at, a hospital on that day (dy1). At time t₁ (day 2), the subject reported the same AE code (AE1), but had a more severe symptom (migraine), and died.

In various embodiments, after repeating processes P1-P3 for subsequent unstructured AE data 40A, the method can further include:

Process P5: comparing the subsequent unstructured reported AE data 40A with the unstructured reported AE data 40 and generating a subject-specific AE report 80 indicating only areas of the subject-specific AE data that have changed between the unstructured reported AE data 40 and the subsequent unstructured reported AE data 40A. With continuing reference to the example table 200 of FIG. 5, this process can include flagging or otherwise indicating (e.g., highlighting, logging, noting, etc.) only the AE data that has changed from one entry to another. In this case, from day 1 to day 2, the subject's headache progressed in severity to a migraine, and that patient went from being admitted to the hospital, to dying. The NLP filter 44 (FIG. 2) can track the progression of this subject over time, and focus only on that unstructured AE data 40, 40A that has changed. The example table 200 in FIG. 2 only provides a small segment of the typical volume of data reported on an hourly, daily or other periodic basis for each subject in a clinical trial. In some cases, hundreds of columns of data are reported for each subject, multiple times per day. Sorting through these columns of data to find meaningful information can be extremely arduous under conventional approaches. The AE data analysis program 30, including the NLP filter 44, is configured to sort through this unstructured AE data 40, 40A and efficiently identify changes over time.

It is understood that subsequent unstructured reported AE data 40A need not necessarily describe an adverse event that occurs at a subsequent (later) time relative to unstructured AE data 40. That is, according to various embodiments, the subsequent unstructured reported AE data 40A could include an update to the original unstructured AE data 40, which may include additional adverse event reporting, different adverse event reporting or identical adverse event reporting. That is, the subsequent unstructured reported AE data 40A may include at least one piece of data that differs from the unstructured reported AE data 40, however, in some cases, the subsequent unstructured reported AE data 40A may include identical (or substantially identical) information as the unstructured reported AE data 40. As noted herein, in various particular embodiments, NLP filter 44 compares the subsequent unstructured reported AE data 40A with the unstructured reported AE data 40 to detect any difference between these data entries, and generate the subject-specific AE report 80.

Additionally, in some embodiments, after generating the subject-specific AE report 80, AE data analysis program 30 can apply NLP filter 44 to any differences in the unstructured reported AE data contained in that AE report 80. That is, where AE report 80 indicates a distinction between the subsequent unstructured reported AE data 40A with the unstructured reported AE data 40, NLP filter 44 can analyze the distinction for a natural language indicator of significance. For example, a distinction in the AE data could include a first description such as “dragging” associated with a first reporting code, and a second description such as “slow” associated with the same reporting code or a different reporting code. NLP filter 44 can be configured to analyze this unstructured AE data to detect natural language characteristics of the input and determine a confidence score for the distinction (or similarity) between the subsequent unstructured reported AE data 40A and the unstructured reported AE data 40. For example, NLP filter 44 can assign a confidence score to the distinctions (or similarities) between the subsequent unstructured reported AE data 40A and the unstructured reported AE data 40 using a conventional F-score approach. In some cases, where applying the NLP filter 44 to the subject-specific AE report 80 indicates an error or other significant discrepancy in the initial reporting codes 58, NLP filter 44 can generate a set of revised (updated) reporting codes based upon the subsequent unstructured reported AE data 40A, and subsequently provide that set of revised (updated) reporting codes for review by the healthcare professional 14 (looping back through processes P1-P5 in FIG. 3, using revised/updated data).

Analyzing Structured AE Data using OCR

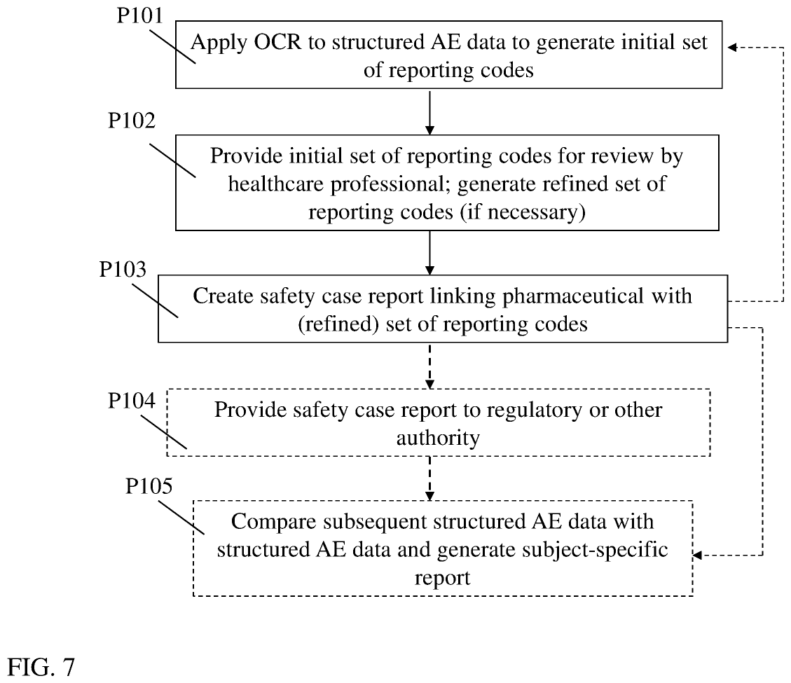

As shown in the data flow diagram 300 of FIG. 6 and the process flow diagram of FIG. 7, in other embodiments, a method can include the following processes:

Process P101: applying optical character recognition (OCR) (e.g., OCR module 46) to the structured reported AE data 42 to generate an initial set of reporting codes 58 for the structured reported AE data 42. As noted herein, in various embodiments, structured (reported) AE data 42 includes information with a high degree of organization, for instance, such that the structured AE data 42 could be readily searchable using simple search engine algorithms or other search operations. This structured AE data 42 could be presented in column/row form or in another format that is easily integrated into a relational database. Like unstructured AE data 40, structured AE data 42 includes data about a sign, symptom or disease of a clinical trial subject. In some particular cases, the structured AE data 42 includes a fillable portable document format (PDF) file, an entry in a spreadsheet, or a fillable text form. OCR module 46 can also include an adverse event thesaurus (AE thesaurus), which may overlap with or include AE thesaurus 50 used in NLP filter 44, or may include a distinct OCR-specific AE thesaurus 60. The OCR-specific AE thesaurus 60 can include correlations between text (and textual phrases) 62 and reporting codes 54.

OCR-specific AE thesaurus 60 can include internally managed connections between textual phrase 62 and AE reporting codes 54, and can be updated continuously based upon results returned from OCR algorithm 64 running structured AE data 42, or manual input from a user (e.g., user 12). Additionally, in various embodiments, OCR-specific AE thesaurus 60 can pull AE reporting codes 54 from an AE reporting code database (DB) 57. AE reporting code DB 57 can include reporting codes from one or more authorities and/or agencies affiliated with reporting of adverse events for pharmaceuticals, vaccines or medical devices. For example, AE reporting code DB 57 can include one or more MedDRA databases, VAERS databases, or other verified databases linking AE reporting codes 54 with particular signs, symptoms or diseases. OCR-specific AE thesaurus 60 can be configured to send updates to AE reporting code DB 57 continuously, periodically or on-demand In various embodiments, a copy of AE reporting code DB 57 can be locally stored at computer system 20, and may be periodically updated. In other cases, AE reporting code DB 57 can be accessed at a central or remote location where it remains continuously, or periodically, updated.

OCR module 46 can include an OCR algorithm 64 configured to perform at least one of the following to the structured reported AE data 42 to generate the initial set of reporting codes 58: a desquew technique, a despeckle technique, a script rule, a text string search, a check mark recognition including a check mark group recognition or a row recognition.

In various embodiments, the initial set of reporting codes 58 generated using the OCR module 46 can include additional data not necessarily included in reporting codes (e.g., initial reporting codes 58) in the approaches utilizing NLP filter 44 (FIG. 2). That is, due to the structured nature of the data 42, 42A, the initial reporting codes 58 in the case of the OCR-based embodiments could include information about data inputs, data formatting, etc., along with structured correlations between data requests (e.g., questions and categories) and inputs (e.g., answers).

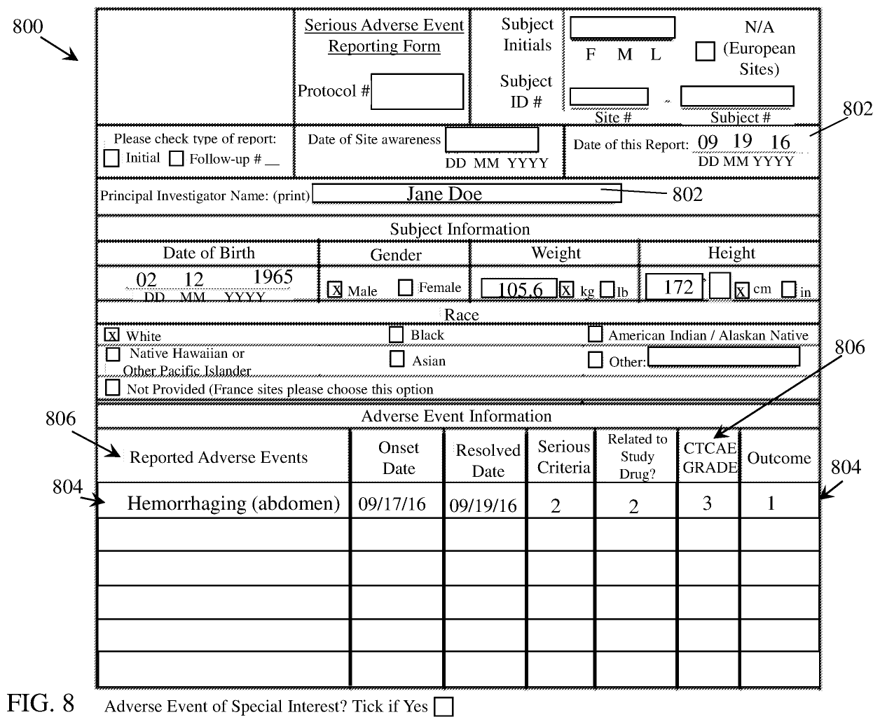

FIG. 8 shows an example depiction of structured reported AE data 42, in the form of a section from a fillable severe adverse event (SAE) reporting form 800, used to report severe adverse events for particular pharmaceutical, vaccine or medical device clinical trials. As shown, the SAE reporting form 800 includes fillable sections 802 for providing information about the subject (patient), such as personal identifying information including subject, height, weight, date-of-birth, race, etc. Fillable sections 802 can also be designed to include event-specific data 804, such as Event Term (e.g., hemorrhaging in the abdomen), Onset Date, Date of Resolution, Serious Criteria, Relationship to Study Drug, Grade (e.g., Common Terminology Criteria for Adverse Events, CTCAE criteria), and Outcome. Fillable sections 802 can be organized by particular headings 806 in the AE data 42. In some cases, particular event-specific data 804 is scored or ranked according to particular reporting criteria. For example, a particular event, such as hemorrhaging in the abdomen, could be classified as “Life-threatening” (score of 2, with 1 being most severe) when it required hospitalization, but did not cause the patient to die. With reference to FIG. 6, the OCR module 46 is configured to identify the terminology in the fillable sections 802, including the event-specific data 804, and select AE reporting codes 54 for that particular event-specific data 804. As noted further herein, OCR module 46 can also flag time-related AE reporting codes 54 for review with subsequent (or prior) structured AE data 42, 42A in order to compare the progress of particular signs, symptoms and diseases for a subject.

OCR module 46 can include an OCR algorithm 64 configured to perform at least one of the following to the structured reported AE data 42 to generate the initial set of reporting codes 58: a desquew technique, a despeckle technique, a script rule, a text string search, a check mark recognition (including a check mark group recognition), a row recognition, etc. In various embodiments, OCR module 46 can obtain the structured reported AE data 42, such as the event-specific (entered) data 804 or other fillable section 802 data (FIG. 8), and rotate, desquew and/or despeckle the AE data 42. OCR module 46 can then apply script rules (e.g., from AE thesaurus 60) based upon the headers, footers and/or images on the intake forms (e.g., the headings 806 in FIG. 8). In various embodiments, OCR module 46 can identify particular terms and data categories using text string search, check mark and check mark group recognition, and/or repeating row recognition (e.g., for tables). Additionally, OCR module 46 can identify a known point or heading (e.g., headings 806) in the AE data 42 as an indicator of input terms or characters, e.g., below, above or on a side of the data input. These terms can be matched with the reporting codes 58 according to OCR module 46 rules (e.g., in OCR algorithm 64). For example, OCR module 46 can identify the heading 806 CTCAE in the SAE reporting form 800 as an indicator of input characters (e.g., numbers 1, 2, 3, etc.) and identify the event-specific data 804 below that heading 806 as the corresponding data input for that particular data category (e.g., CTCAE grade of “3” in this case).

Following process P101, in some cases, process P102 can include: providing the initial set of reporting codes 58 for review by a healthcare professional 14, to either verify each of the reporting codes 58 or modify at least one of the reporting codes 58, and generating a refined set of reporting codes 70 based upon the review. In various embodiments, providing the initial set of reporting codes 58 includes displaying, sending or presenting an editable version of the initial set of reporting codes 58 to the healthcare professional 14. Generating the refined set of reporting codes 70 can include incorporating at least one modification from the initial set of codes 58 based upon an edit made by the healthcare professional 14. This process may be performed in a substantially similar manner as process P2 described with reference to FIG. 3.

After generating the refined set of reporting codes 70, process P103 can include: creating a safety case report 72 linking the pharmaceutical, vaccine or medical device with the refined set of reporting codes 70. The safety case report 72 can include individual subject reporting codes, as well as codes sorted according to severity, frequency, geography or any other pertinent sorting/grouping criteria. Additionally, safety case report 72 can include a narrative of the course of the (adverse) event, a medical history of the subject, concomitant medications with the pharmaceutical, an assessment (e.g., from event reporter) of causality, and/or an assessment (e.g., from event reporter or other source) as to whether the event is expected as per the product label.

In various embodiments, the process can further include:

Process P104: providing the safety case report 72 to a regulatory authority or other authority. This process may be performed in a substantially similar manner as process P4 described with reference to FIG. 3.

Additionally, as shown in FIG. 7, in some cases, processes P101-P103 can be repeated for subsequent structured reported AE data 42A. This subsequent structured reported AE data 42A, along with the structured AE data 42 each include subject-specific AE data about a set of trial subjects. In some cases, the subsequent structured reported AE data 42A describes a sign, symptom or disease of the set of subjects in response to the pharmaceutical, vaccine or medical device at a time (t₁) later than the structured reported AE data 42 (from time t₀) about the subject. As described herein, FIG. 5 shows an example table 200 of a portion of subject-specific AE data (i.e., data about a particular trial subject).

In various embodiments, after repeating processes P101-P103 for subsequent structured AE data 42A, the method can further include:

Process P105: comparing the subsequent structured reported AE data 42A with the structured reported AE data 42 and generating a subject-specific AE report 80 indicating only areas of the subject-specific AE data that have changed between the structured reported AE data 42 and the subsequent structured reported AE data 42A. This process is performed similarly to process P5 described with reference to FIG. 3 and the example table 200 of FIG. 5.

Analyzing Unstructured AE Data using NLP and Data Visualization (DV)

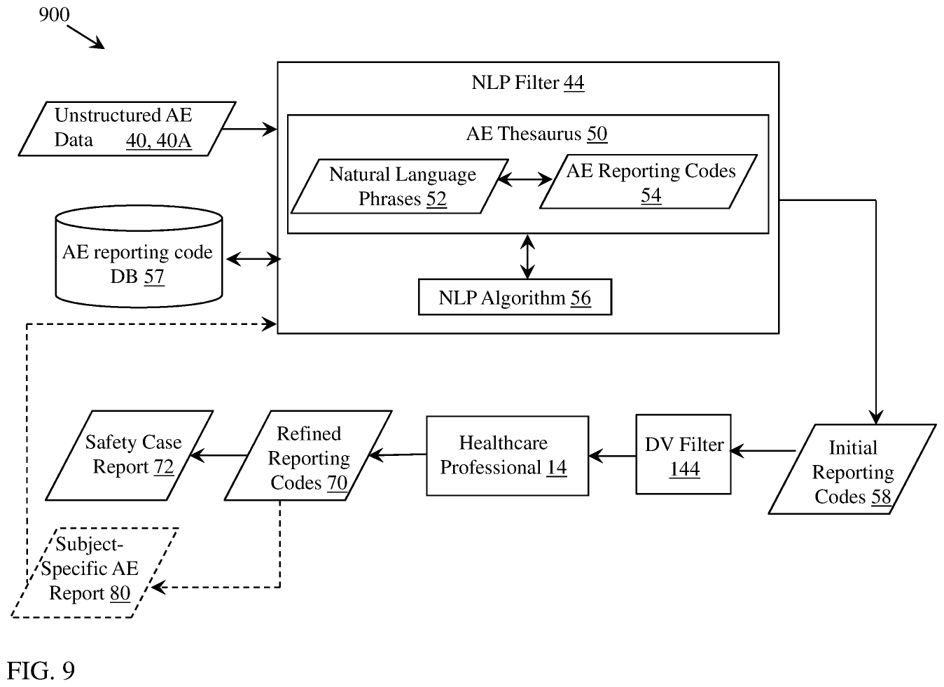

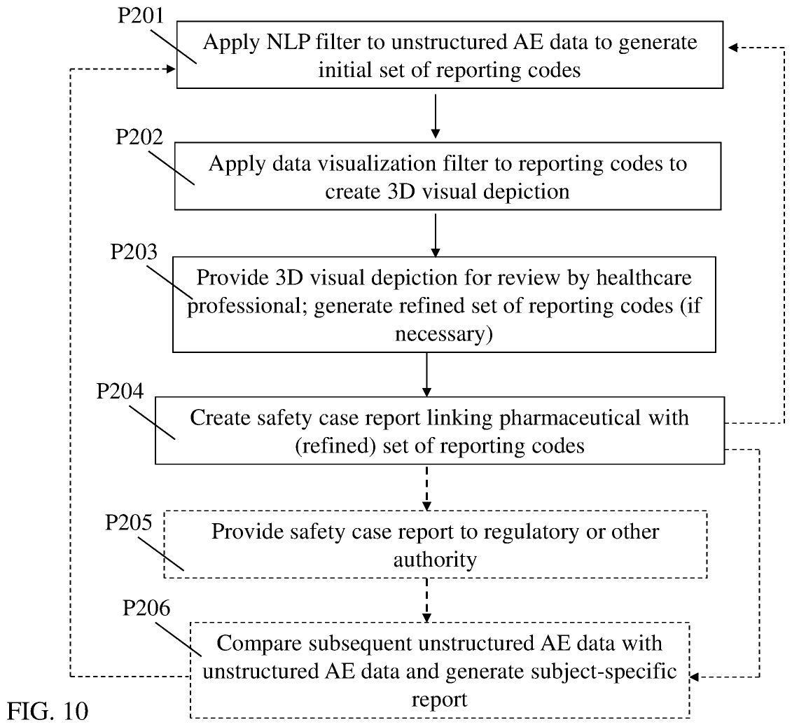

As shown in the data flow diagram of FIG. 9 and the process flow diagram 900 of FIG. 10, in other embodiments, a method can include the following processes:

Process P201: applying natural language processing (NLP) filter 44 to the unstructured reported AE data 40 to generate an initial set of reporting codes 58 for that unstructured reported AE data 40 (see process P1 above).

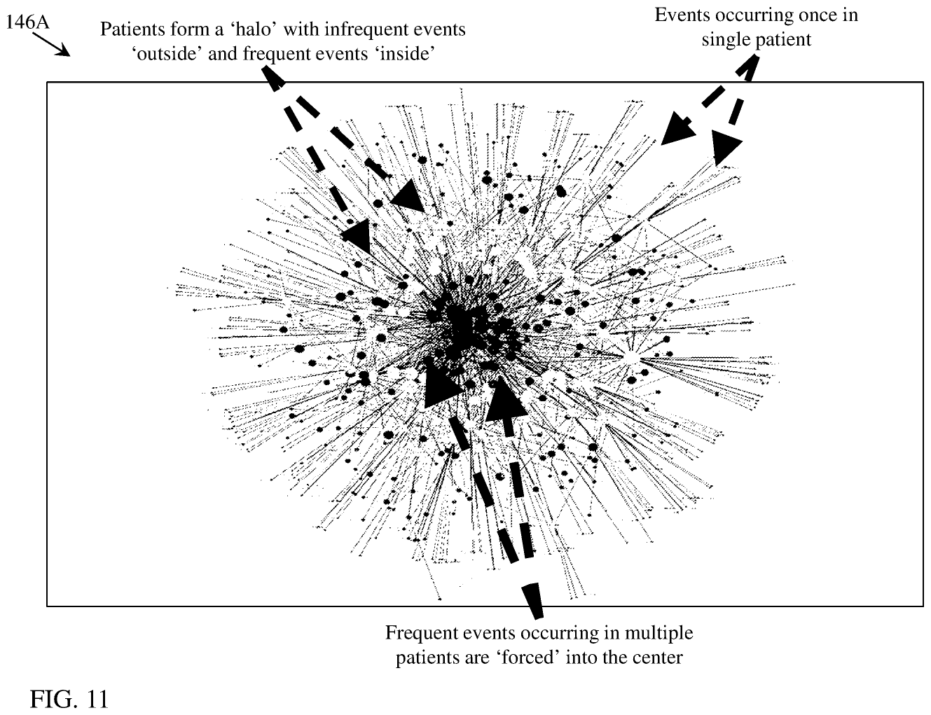

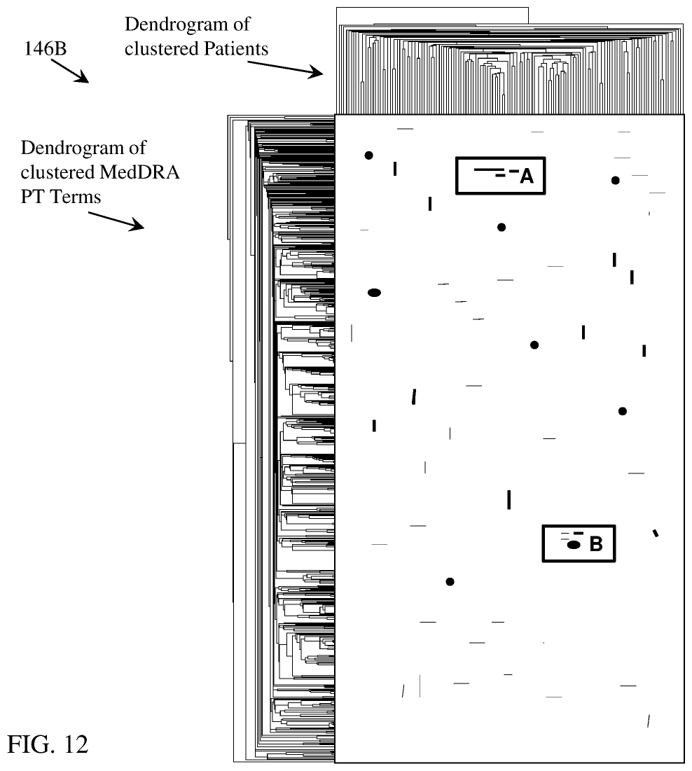

Following process P101, process P202 can include: applying a data visualization filter (DV filter) 144 to the set of reporting codes 58 to create a (e.g., three-factor, or three-dimensional (3D)) visual depiction 146 of the reporting codes 58 for the unstructured reported AE data 40. FIGS. 10 and 11 show example visual depictions 146A, 146B of reporting codes 58 according to embodiments of the disclosure. FIG. 11 shows a three-dimensional visual depiction (e.g., a web or multi-dimensional node map) 146A of reporting codes 58 representing events (e.g., adverse events). As shown, in some cases, a “halo” effect depicts infrequent events along an outer arc and more frequent events along an inner arc. Outlying events, such as those occurring once in a single patient, sit at the outer edges of the 3D depiction 146A. Conversely, higher-frequency events are concentrated in the central region of the 3D depiction 146A. Color may be used to indicate distinctions in events and trends, for example, contrasting colors or variations in intensity may demonstrate distinctions in event frequency. FIG. 12 illustrates another visual depiction 146B, which includes a “heat map” that uses contrasting color (e.g., red or orange, with black background) to indicate the intensity and frequency of particular events and reporting codes 58, e.g., in clusters. As shown, the heat map is correlated with a dendrogram (tree structure) illustrating a hierarchical structure to the reporting codes 58. Clusters A and B are shown to illustrate two distinct high-frequency events at distinct hierarchies (e.g., A having a higher importance than B).

Following process P202, process P203 can include: providing the (e.g., three-factor, or 3D) visual depiction 146 for review by healthcare professional 14, to either verify each of the reporting codes 58 or modify at least one of the reporting codes 58, and generating a refined set of reporting codes 70 based upon the review. This process can be performed substantially similarly to process P2 described with respect to FIG. 3. However, in the case of reviewing the visual depiction 146, the healthcare professional 14 (e.g. human user or computing device) can rely upon visual trends in the display or depiction of the reporting codes 58 that may not be as easily grasped (or grasped at all) in conventional data reporting and review. For example, in contrast to review of a spreadsheet of data, the visualization approach can more clearly identify clusters of data (e.g., codes, patients, etc.) or particular trends in that data. Additionally, some visual depictions 146 rely upon the odds ratio of statistical filtering, which enhances identification of trends by quantifying how strongly the presence or absence of a first property (property A) is associated with the presence or absence of second property (property B) in a given population or dataset. According to various embodiments, the visual depiction 146 can utilize variables that are set independently of reporting codes 58 or dictionary terms in order to correlate properties of subject(s) (e.g., subject history, other medications, etc.), pharmaceutical(s), vaccine(s), medical device(s), time frame(s), etc.

Following process P203, process P204 can include: creating a safety case report 72 linking the pharmaceutical, vaccine or medical device with the refined set of reporting codes 70. This process may be performed in a substantially similar manner as process P4 described with reference to FIG. 3.

In various embodiments, the process can further include:

Process P205: providing the safety case report 72 to a regulatory authority or other authority. This process may be performed in a substantially similar manner as process P4 described with reference to FIG. 3.

Additionally, as shown in FIG. 10, in some cases, processes P201-P204 can be repeated for subsequent unstructured reported AE data 40A. This subsequent unstructured reported AE data 40A, along with the unstructured AE data 40 each include subject-specific AE data about a set of trial subjects. In some cases, the subsequent unstructured reported AE data 40A describes a sign, symptom or disease of the set of subjects in response to the pharmaceutical, vaccine or medical device at a time (t₁ ) later than the unstructured reported AE data 40 (from time t₀) about the subject. FIG. 5 shows an example tabulated depiction of a portion of subject-specific AE data (i.e., data about a particular trial subject).

In various embodiments, after repeating processes P201-P204 for subsequent unstructured AE data 40A, the method can further include:

Process P206: comparing the subsequent unstructured reported AE data 40A with the unstructured reported AE data 40 and generating a subject-specific AE report 80 indicating only areas of the subject-specific AE data that have changed between the unstructured reported AE data 40 and the subsequent unstructured reported AE data 40A. This process is performed similarly to process P5 described with reference to FIG. 3 and the example table 200 of FIG. 5.

As noted herein, it is understood that subsequent unstructured reported AE data 40A need not necessarily describe an adverse event that occurs at a subsequent (later) time relative to unstructured AE data 40. That is, according to various embodiments, the subsequent unstructured reported AE data 40A could include an update to the original unstructured AE data 40, which may include additional adverse event reporting, different adverse event reporting or identical adverse event reporting. That is, the subsequent unstructured reported AE data 40A may include at least one piece of data that differs from the unstructured reported AE data 40, however, in some cases, the subsequent unstructured reported AE data 40A may include identical (or substantially identical) information as the unstructured reported AE data 40. As noted herein, in various particular embodiments, NLP filter 44 compares the subsequent unstructured reported AE data 40A with the unstructured reported AE data 40 to detect any difference between these data entries, and generate the subject-specific AE report 80.

Additionally, in some embodiments, after generating the subject-specific AE report 80, AE data analysis program 30 can apply NLP filter 44 to any differences in the unstructured reported AE data contained in that AE report 80. That is, where AE report 80 indicates a distinction between the subsequent unstructured reported AE data 40A and the unstructured reported AE data 40, NLP filter 44 can analyze the distinction for a natural language indicator of significance. For example, a distinction in the AE data could include a first description such as “dragging” associated with a first reporting code, and a second description such as “slow” associated with the same reporting code or a different reporting code. NLP filter 44 can be configured to analyze this unstructured AE data to detect natural language characteristics of the input and determine a confidence score for the distinction (or similarity) between the subsequent unstructured reported AE data 40A and the unstructured reported AE data 40. In some cases, where applying the NLP filter 44 to the subject-specific AE report 80 indicates an error or other significant discrepancy in the initial reporting codes 58, NLP filter 44 can generate a set of revised (updated) reporting codes based upon the subsequent unstructured reported AE data 40A, and subsequently provide that set of revised (updated) reporting codes for review by the healthcare professional 14 (looping back through processes P201-P206 in FIG. 10, using the revised/updated data).

Aspects disclosed herein provide several features not found in conventional adverse event analysis and reporting systems. For example, both structured adverse event data and unstructured adverse event data can be efficiently and effectively processed using the various approaches, systems and computer program products described herein. Further, the embodiments described herein can track the adverse event progress of particular trial subjects over time, allowing for further insight to the effects of particular pharmaceuticals, vaccines and/or medical devices. Additionally, when compared with conventional approaches, these embodiments can provide improved data (including visualized data) to healthcare professionals for analysis and review, thereby streamlining the process of verifying adverse event reporting.

While shown and described herein as a method and system for analyzing adverse event data, it is understood that aspects of the disclosure further provide various alternative embodiments. For example, in one embodiment, the disclosure provides a computer program fixed in at least one computer-readable medium, which when executed, enables a computer system to analyze adverse event data. To this extent, the computer-readable medium includes program code, such as AE data analysis program 30 (FIG. 1), which enables a computer system to implement some or all of a process described herein. It is understood that the term “computer-readable medium” comprises one or more of any type of tangible medium of expression, now known or later developed, from which a copy of the program code can be perceived, reproduced, or otherwise communicated by a computing device. For example, the computer-readable medium can comprise: one or more portable storage articles of manufacture; one or more memory/storage components of a computing device; paper; and/or the like.

In another embodiment, the disclosure provides a method of providing a copy of program code, such as AE data analysis program 30 (FIG. 1), which enables a computer system to implement some or all of a process described herein. In this case, a computer system can process a copy of the program code to generate and transmit, for reception at a second, distinct location, a set of data signals that has one or more of its characteristics set and/or changed in such a manner as to encode a copy of the program code in the set of data signals. Similarly, an embodiment of the disclosure provides a method of acquiring a copy of the program code, which includes a computer system receiving the set of data signals described herein, and translating the set of data signals into a copy of the computer program fixed in at least one computer-readable medium. In either case, the set of data signals can be transmitted/received using any type of communications link.

In still another embodiment, the disclosure provides a method of generating an AE data analysis program 30. In this case, a computer system, such as computer system 20 (FIG. 1), can be obtained (e.g., created, maintained, made available, etc.) and one or more components for performing a process described herein can be obtained (e.g., created, purchased, used, modified, etc.) and deployed to the computer system. To this extent, the deployment can comprise one or more of: (1) installing program code on a computing device; (2) adding one or more computing and/or I/O devices to the computer system; (3) incorporating and/or modifying the computer system to enable it to perform a process described herein; and/or the like.

It is understood that aspects of the disclosure can be implemented as part of a business method that performs a process described herein on a subscription, advertising, and/or fee basis. That is, a service provider could offer to provide an adverse event data analysis program as described herein. In this case, the service provider can manage (e.g., create, maintain, support, etc.) a computer system, such as computer system 20 (FIG. 1), that performs a process described herein for one or more customers. In return, the service provider can receive payment from the customer(s) under a subscription and/or fee agreement, receive payment from the sale of advertising to one or more third parties, and/or the like.

In any case, the technical effect of the various embodiments of the disclosure, including, e.g., AE data analysis program 30, is to analyze adverse event data in order to generate a safety report (e.g., safety case report 72). In various embodiments, the technical effect of the of the AE data analysis program 30 is to provide an improved mechanism for generating safety reports (e.g., safety case report 72) using one or more filter(s) or modules tailored to the format of the AE data.

The foregoing description of various aspects of the disclosure has been presented for purposes of illustration and description. It is not intended to be exhaustive or to limit the disclosure to the precise form disclosed, and obviously, many modifications and variations are possible. Such modifications and variations that may be apparent to an individual in the art are included within the scope of the disclosure as defined by the accompanying claims.

We claim:

1. A computer-implemented method for analyzing unstructured reported adverse event (AE) data about a pharmaceutical, a vaccine or a medical device as reported by a set of trial subjects to enhance accuracy in safety case reporting for the pharmaceutical, vaccine or medical device, the method comprising:
applying a natural language processing (NLP) filter to the unstructured reported AE data, wherein the unstructured reported AE data comprises subject-specific AE data about the set of trial subjects, and wherein the unstructured reported AE data does not have a pre-defined data model or is not organized in a predefined manner;
generating an initial set of reporting codes for the unstructured reported AE data from the filtered unstructured reported AE data;
applying a data visualization filter to the initial set of reporting codes to create a visual depiction of the initial set of reporting codes for the unstructured reported AE data on a physical user interface, wherein the visual depiction includes a three-dimensional data map or a cluster map of reporting codes showing interconnections between particular AE signs, symptoms and/or diseases and particular subjects or their groups, or wherein the visual depiction includes a heat map which uses contrasting color to indicate an intensity and frequency of occurrences of reporting codes in clusters;
providing the visual depiction for review by the healthcare professional along with the initial set of reporting codes for review by a healthcare professional, to either verify each of the reporting codes or modify at least one of the reporting codes, and generating a refined set of reporting codes based upon the review; and
creating a safety case report linking the pharmaceutical, the vaccine or the medical device with the refined set of reporting codes as verified or modified by the healthcare professional,
wherein applying the NLP filter, generating the initial set of reporting codes, applying the data visualization filter, and providing the initial set of reporting codes for review by the healthcare professional enhances accuracy in the safety case reporting by mitigating syntax-based mischaracterization of the unstructured reported AE data and mitigating time spent in processing the unstructured reported AE data.

2. The computer-implemented method of claim 1, further comprising:
providing the safety case report to a regulatory authority or other authority,
wherein the regulatory authority or other authority requires immediate reporting of serious adverse events (SAEs) for the set of trial subjects, wherein the unstructured reported AE data comprises subject-specific AE data about at least one SAE for the set of trial subjects, and
wherein applying the NLP filter, generating the initial set of reporting codes, and providing the initial set of reporting codes for review by the healthcare professional enhances accuracy in reporting the at least one SAE by mitigating syntax-based mischaracterization of the unstructured reported AE data about the at least one SAE and mitigating time spent in processing the unstructured reported AE data about the at least one SAE.

3. The computer-implemented method of claim 2, wherein the visual depiction of the initial set of reporting codes uses variables that are set independently of the reporting codes or dictionary terms to correlate properties of at least two of:
i) the set of trial subjects, ii) the pharmaceutical, the vaccine or the medical device, and iii) a time frame in which the unstructured reported AE data was reported.

4. The computer-implemented method of claim 1, wherein providing the initial set of reporting codes includes displaying, sending or presenting an editable version of the initial set of reporting codes to the healthcare professional, wherein generating the refined set of reporting codes includes incorporating at least one modification from the initial set of reporting codes based upon an edit made by the healthcare professional.

5. The computer-implemented method of claim 1, further comprising:
repeating the applying of the natural language processing (NLP) filter, the providing of the initial set of reporting codes for review, and the creating of the safety case report for subsequent unstructured reported AE data, wherein the unstructured reported AE data and the subsequent unstructured reported AE data each include subject-specific AE data about a set of trial subjects; and
comparing the subsequent unstructured reported AE data with the unstructured reported AE data and generating a subject-specific AE report indicating only areas of the subject-specific AE data that have changed between the unstructured reported AE data and the subsequent unstructured reported AE data,
wherein the subsequent unstructured reported AE data describes a sign, symptom or disease of the set of subjects in response to the pharmaceutical, the vaccine or the medical device at a time later than the unstructured reported AE data about the subject, and wherein generating the subject-specific AE report that indicates only the areas of the subject-specific AE data that have changed between the unstructured reported AE data and the subsequent unstructured reported AE data enhances accuracy in the safety case reporting by mitigating syntax-based mischaracterization of the subsequent unstructured reported AE data and mitigating time spent in processing the subsequent unstructured reported AE data.

6. The computer-implemented method of claim 5, wherein the method further comprises:
applying the natural language processing (NLP) filter to the subject-specific AE report to generate an updated set of reporting codes for the unstructured reported AE data;
providing the updated set of reporting codes for review by the healthcare professional, to either verify each of the updated set of reporting codes or modify at least one of the updated set of reporting codes, and generating an updated refined set of reporting codes based upon the updated review; and
creating an updated safety case report linking the pharmaceutical, the vaccine or the medical device with the updated refined set of reporting codes.

7. The computer-implemented method of claim 5, wherein the unstructured reported AE data includes data about a sign, symptom or disease of a clinical trial subject, wherein the NLP filter is further configured to assign a confidence score to the assignment of the unstructured reported AE data with the initial set of reporting codes,
wherein the method further comprises:
flagging reporting codes in the initial set of reporting codes that are assigned a confidence score below a threshold confidence score; and
requiring additional review or special review of the flagged reporting codes by the healthcare professional, via the physical user interface depicting the visual depiction of the initial set of reporting codes, prior to creating the safety case report.

8. The computer-implemented method of claim 7, wherein the unstructured reported AE data includes a social media post.

9. The computer-implemented method of claim 1, wherein the healthcare professional is one of a human being or a programmable computing device including a logic engine.

10. The computer-implemented method of claim 1, wherein the unstructured reported AE data includes at least one of: a string of text, a social media post, or a voice to text conversion of an audio recording,
wherein the NLP filter includes an adverse event thesaurus (AE thesaurus) including correlations between natural language phrases and AE reporting codes,
wherein the NLP filter includes an NLP algorithm configured to perform at least one of the following to the unstructured reported AE data to generate the initial set of reporting codes: English slot grammar (ESG) parsing, entity detection, sense disambiguation, aggregation, declarative rule generation, relationship extraction, sentence breaking or word segmentation, and
wherein the AE thesaurus is configured to add new natural language phrases and correlations with AE reporting codes iteratively, and wherein the AE thesaurus is manually updateable.

11. A computer-implemented method for analyzing structured reported adverse event (AE) data about a pharmaceutical, a vaccine or a medical device as reported by a set of trial subjects to enhance accuracy in safety case reporting for the pharmaceutical, vaccine or medical device, the method comprising:
applying optical character recognition (OCR) to the structured reported AE data, wherein the structured reported AE data comprises subject-specific AE data about the set of trial subjects;
generating an initial set of reporting codes for the structured reported AE data from the OCR-applied structured reported AE data;
applying a data visualization filter to the initial set of reporting codes to create a visual depiction of the initial set of reporting codes for the structured reported AE data on a physical user interface,
wherein the visual depiction includes a three-dimensional data map or cluster map of reporting codes showing interconnections between particular AE signs, symptoms and/or diseases and particular subjects or their groups, or wherein the visual depiction includes a heat map which uses contrasting color to indicate an intensity and frequency of occurrences of reporting codes in clusters;
providing the visual depiction along with the initial set of reporting codes for review by a healthcare professional, to either verify each of the reporting codes or modify at least one of the reporting codes, and generating a refined set of reporting codes based upon the review; and
creating a safety case report linking the pharmaceutical, the vaccine or the medical device with the refined set of reporting codes as verified or modified by the healthcare professional,
wherein applying the OCR, generating the initial set of reporting codes, applying the data visualization filter, and providing the initial set of reporting codes for review by the healthcare professional enhances accuracy in the safety case reporting by mitigating mischaracterization of the structured reported AE data and mitigating time spent in processing the structured reported AE data.

12. The computer-implemented method of claim 11, further comprising:
providing the safety case report to a regulatory authority or other authority,
wherein the regulatory authority or other authority requires immediate reporting of serious adverse events (SAEs) for the set of trial subjects, wherein the structured reported AE data comprises subject-specific AE data about at least one SAE for the set of trial subjects, and
wherein applying the OCR, generating the initial set of reporting codes, and providing the initial set of reporting codes for review by the healthcare professional enhances accuracy in reporting the at least one SAE by mitigating mischaracterization of the structured reported AE data about the at least one SAE and mitigating time spent in processing the structured reported AE data about the at least one SAE.

13. The computer-implemented method of claim 11, wherein providing the initial set of reporting codes includes displaying, sending or presenting an editable version of the initial set of reporting codes to the healthcare professional, and
wherein generating the refined set of reporting codes includes incorporating at least one modification from the initial set of reporting codes based upon an edit made by the healthcare professional.

14. The computer-implemented method of claim 11, further comprising:
repeating the applying of the OCR, the providing of the initial set of reporting codes for review, and the creating of the safety case report for subsequent structured reported AE data, wherein the structured reported AE data and the subsequent structured reported AE data each include subject-specific AE data about a set of trial subjects;
comparing the subsequent structured reported AE data with the structured reported AE data and generating a subject-specific AE report indicating only areas of the subject-specific AE data that have changed between the structured reported AE data and the subsequent structured reported AE data,
wherein the subsequent structured reported AE data describes a sign, symptom or disease of the set of subjects in response to the pharmaceutical, the vaccine or the medical device at a time later than the structured reported AE data about the subject,
wherein generating the subject-specific AE report that indicates only the areas of the subject-specific AE data that have changed between the structured reported AE data and the subsequent structured reported AE data enhances accuracy in the safety case reporting by mitigating syntax-based mischaracterization of the subsequent structured reported AE data and mitigating time spent in processing the subsequent structured reported AE data;
applying a natural language processing (NLP) filter to the subject-specific AE report to generate an updated set of reporting codes for the unstructured reported AE data;
providing the updated set of reporting codes for review by the healthcare professional, to either verify each of the updated set of reporting codes or modify at least one of the updated set of reporting codes, and generating an updated refined set of reporting codes based upon the updated review; and
creating an updated safety case report linking the pharmaceutical, the vaccine or the medical device with the updated refined set of reporting codes.

15. The computer-implemented method of claim 11, wherein the healthcare professional is a human being.

16. The computer-implemented method of claim 11, wherein the healthcare professional is a programmable computing device including a logic engine.

17. The computer-implemented method of claim 11, wherein the structured reported AE data includes data about a sign, symptom or disease of a clinical trial subject,
wherein the structured reported AE data includes at least one of: a fillable portable document format (PDF) file, an entry in a spreadsheet or a fillable text form.

18. The computer-implemented method of claim 11, wherein the OCR is performed by an OCR module including an adverse event thesaurus (AE thesaurus) including correlations between text and AE reporting codes.

19. The computer-implemented method of claim 18, wherein the OCR module includes an OCR algorithm configured to perform at least one of the following to the structured reported AE data to generate the initial set of reporting codes: a desquew technique, a despeckle technique, a script rule, a text string search, a check mark recognition including a check mark group recognition or a row recognition, and
wherein the AE thesaurus is configured to add new textual terms and correlations with AE reporting codes iteratively, and wherein the AE thesaurus is manually updateable.

20. The computer-implemented method of claim 11, wherein the visual depiction of the initial set of reporting codes uses variables that are set independently of the reporting codes or dictionary terms to correlate properties of at least two of:
i) the set of trial subjects, ii) the pharmaceutical, the vaccine or the medical device, and iii) a time frame in which the structured reported AE data was reported.
